(19) United States
(12) Patent Application Publication
(10) Pub. No.: US 2022/0233569 A1
(43) Pub. Date: Jul. 28, 2022

(54) Methods Of Improving Health With Apolipoprotein E (APOE) Inhibitors

(71) Applicant: Regeneron Pharmaceuticals, Inc., Tarrytown, NY (US)

(72) Inventors: Jan Freudenberg, Tarrytown, NY (US); Christian Benner, Tarrytown, NY (US)

(21) Appl. No.: 17/584,642

(22) Filed: Jan. 26, 2022

Related U.S. Application Data
(60) Provisional application No. 63/141,503, filed on Jan. 26, 2021.

(51) Int. Cl. A61K 31/7088; A61K 38/46; C12Q 1/6883; A61K 38/17; C07K 16/18

(52) U.S. Cl. CPC A61K 31/7088; A61K 38/465; C12Q 1/6883; C12Q 2600/158; C07K 16/18; C12Q 2600/118; A61K 38/177

(57) ABSTRACT

Methods of increasing longevity and/or inducing healthy aging, and methods of identifying subjects having an increased risk of lower longevity and/or developing one or more age-related diseases are disclosed herein.

### REFERENCE TO SEQUENCE LISTING

This application includes a Sequence Listing submitted electronically as a text file named 18923806101SEQ, created on Jan. 23, 2022, with a size of 43 kilobytes. The Sequence Listing is incorporated herein by reference.

### FIELD

The present disclosure relates generally to increasing longevity and/or inducing healthy aging in a subject by administering APOE inhibitors, and methods of identifying subjects having an expectation of lower longevity and/or developing one or more age-related diseases, such as Alzheimer's disease or coronary artery disease.

### BACKGROUND

Aging is the gradual loss of function and deterioration at the cellular, tissue, and organ level, leading to increased susceptibility to disease and external stressors, and eventually death. All organisms age, but the effects of aging can be slowed, minimized, and/or manipulated. Numerous experiments have shown the ability to increase maximal lifespan as well as healthspan with decreased susceptibility to other age-related pathologies. Aging interventions examined to date have included environmental manipulation such as calorie restriction, small molecule drugs such as rapamycin, and genetic manipulations accomplished through the creation of transgenic animals, such as the Ames and Snell dwarf mice. While these studies have led to a greater understanding of the mechanisms involved in aging, they are not amenable to translation to aging human populations.

Treatment of age-related diseases is critical for an increase in lifespan and improvement in the quality of life in the advanced-age population. Several genes that are involved in aging and lifespan control have been proposed. These so-called longevity genes are often involved in pathways that guarantee better survival under conditions such as food deprivation, cold, or other types of environmental stress (Porcu and Chiarugi, Trends in Pharmacolog. Sciences, 2005, 26, 94).

Apolipoprotein E (APOE) is an apolipoprotein, a protein associating with lipid particles, that mainly functions in lipoprotein-mediated lipid transport between organs via the plasma and interstitial fluids. APOE is a core component of plasma lipoproteins and is involved in their production, conversion, and clearance. Apoliproteins are amphipathic molecules that interact both with lipids of the lipoprotein particle core and the aqueous environment of the plasma. As such, APOE associates with chylomicrons, chylomicron remnants, very low density lipoproteins (VLDL), and intermediate density lipoproteins (IDL), but shows a preferential binding to high-density lipoproteins (HDL). It also binds a wide range of cellular receptors including the LDL receptor/LDLR, the LDL receptor-related proteins LRP1, LRP2 and LRP8, and the very low-density lipoprotein receptor/VLDLR that mediate the cellular uptake of the APOE-containing lipoprotein particles. Finally, APOE also has a heparin-binding activity and binds heparan-sulfate proteoglycans on the surface of cells, a property that supports the capture and the receptor-mediated uptake of APOE-containing lipoproteins by cells. A function of APOE is to mediate lipoprotein clearance through the uptake of chylomicrons, VLDLs, and HDLs by hepatocytes. APOE is also involved in the biosynthesis by the liver of VLDLs as well as their uptake by peripheral tissues ensuring the delivery of triglycerides and energy storage in muscle, heart, and adipose tissues. By participating in the lipoprotein-mediated distribution of lipids among tissues, APOE plays a critical role in plasma and tissues lipid homeostasis. APOE is also involved in two steps of reverse cholesterol transport, the HDLs-mediated transport of cholesterol from peripheral tissues to the liver, and thereby plays an important role in cholesterol homeostasis. First, it is functionally associated with ABCA1 in the biogenesis of HDLs in tissues. Second, it is enriched in circulating HDLs and mediates their uptake by hepatocytes. APOE also plays an important role in lipid transport in the central nervous system, regulating neuron survival and sprouting. APOE is also involved in innate and adaptive immune responses, controlling for instance the survival of myeloid-derived suppressor cells. APOE, may also play a role in transcription regulation through a receptor-dependent and cholesterol-independent mechanism, that activates MAP3K12 and a non-canonical MAPK signal transduction pathway that results in enhanced AP-1-mediated transcription of APP.

### SUMMARY

The present disclosure provides methods of increasing longevity in a subject, the method comprising administering an APOE inhibitor to the subject.

The present disclosure also provides methods of inducing healthy aging in a subject, the method comprising administering an APOE inhibitor to the subject.

The present disclosure also provides methods of increasing longevity or inducing healthy aging in a subject, wherein the subject is at risk of decreased longevity and/or is suffering from one or more age-related diseases, the method comprising the steps of: determining whether the subject has an APOE predicted loss-of-function variant nucleic acid molecule by: obtaining or having obtained a biological sample from the subject; and performing or having performed a genotyping assay on the biological sample to determine if the subject has a genotype comprising the APOE predicted loss-of-function variant nucleic acid molecule; and when the subject is APOE reference, then administering or continuing to administer to the subject an APOE inhibitor in a standard dosage amount; and when the subject is heterozygous for an APOE predicted loss-of-function variant, then administering or continuing to administer to the subject the APOE inhibitor in an amount that is lower than a standard dosage amount; wherein the presence of a genotype having the APOE predicted loss-of-function variant nucleic acid molecule indicates the subject has a reduced risk of lower longevity and/or developing one or more age-related diseases.

The present disclosure also provides methods of identifying a subject having an increased risk for lower longevity and/or developing one or more age-related diseases, wherein the method comprises: determining or having determined the presence or absence of an APOE predicted loss-of-function variant nucleic acid molecule in a biological sample obtained from the subject; wherein: when the subject is APOE reference, then the subject has an increased risk for lower longevity and/or developing one or more age-related diseases; and when the subject is heterozygous for an APOE predicted loss-of-function variant nucleic acid molecule or homozygous for an APOE predicted loss-of-function variant nucleic acid molecule, then the subject has a decreased risk for lower longevity and/or developing one or more age-related diseases.

The present disclosure also provides APOE inhibitors for use in increasing longevity and/or inducing healthy aging in a subject having an APOE genomic nucleic acid molecule having a nucleotide sequence comprising a thymine at a position corresponding to position 501 according to SEQ ID NO:2, or the complement thereof.

### BRIEF DESCRIPTION OF THE DRAWINGS

The accompanying FIGURES, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows association results conditional on rs429358 for variants across the APOE locus with uric acid (UA), cystatin-C(CYSC), C-reactive protein (CRP), high-density lipoprotein (HDL), low-density-lipoprotein (LDL), and triglycerides (TRIG). The −log 10(p-values) are presented on the y-axis and variant positions are on the x-axis. For LDL the absolute values of the t-statistic are printed on the y-axis instead of p-values. The color of points shows the estimated linkage disequilibrium with rs1065853.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

A variant adjacent to the APOE gene associated with several blood biomarkers in subjects has been identified in accordance with the present disclosure. For example, SNP rs1065853 (located at chr19:44909976 according to GRCh38/hg38 human genome assembly), a genetic alteration in the non-coding region that changes the guanine nucleotide of position 501 in the human APOE reference genomic nucleic acid molecule (see, SEQ ID NO:1) to thymine, has been observed to indicate that the subject having such an alteration may have increased longevity, healthy aging, and a decreased risk of developing one or more age-related diseases, such as cardiovascular disease, diabetes, atherosclerosis, obesity, cancer, infection, immunosenescence, and neurological disorders. It is believed that no specific non-coding region variants of the APOE locus have any demonstrated causal effect on increased longevity, healthy aging, and decreased risk of age-related diseases and that all the known associations of APOE are instead caused by its coding variants. Altogether, the genetic analyses described herein surprisingly indicate that the APOE gene and, in particular, a variant in the non-coding region adjacent to the APOE genomic nucleic acid molecule, is a cause of increased longevity, healthy aging, and a decreased risk of developing one or more age-related diseases (e.g., that this noncoding variant is causing the APOE associations with longevity and related traits instead of or together with the correlated coding APOE Arg176Cys variant as previously believed). Therefore, subjects that are APOE reference that have an increased risk for lower longevity and/or developing one or more age-related diseases may be treated such that longevity is increased, healthy aging is induced, and/or development of age-related diseases is prevented or delayed, the symptoms thereof are reduced, and/or development of symptoms is repressed. Accordingly, the present disclosure provides methods of leveraging the identification of such variants in subjects to identify or stratify risk in such subjects of having decreased longevity and of developing age-related diseases, or to diagnose subjects as having an increased risk of lower longevity and/or developing one or more age-related diseases, such that subjects at risk or subjects with active disease may be treated accordingly.

For purposes of the present disclosure, any particular subject can be categorized as having one of three APOE genotypes: i) APOE reference; ii) heterozygous for an APOE predicted loss-of-function variant; or iii) homozygous for an APOE predicted loss-of-function variant. A subject is APOE reference when the subject does not have a copy of an APOE predicted loss-of-function variant nucleic acid molecule. A subject is heterozygous for an APOE predicted loss-of-function variant when the subject has a single copy of an APOE predicted loss-of-function variant nucleic acid molecule. An APOE predicted loss-of-function variant nucleic acid molecule is any APOE nucleic acid molecule (such as, a genomic nucleic acid molecule, an mRNA molecule, or a cDNA molecule) resulting in a partial loss-of-function, a complete loss-of-function, a predicted partial loss-of-function, or a predicted complete loss-of-function of APOE activity. An exemplary APOE predicted loss-of-function variant nucleic acid molecule is an APOE genomic nucleic acid molecule having an adjacent non-coding region comprising the SNP rs1065853. A subject is homozygous for an APOE predicted loss-of-function variant nucleic acid molecule when the subject has two copies of an APOE predicted loss-of-function variant nucleic acid molecule.

For subjects that are genotyped or determined to be APOE reference, such subjects have decreased longevity and an increased risk of developing one or more age-related diseases, such as cardiovascular disease, diabetes, atherosclerosis, obesity, cancer, infection, immunosenescence, and neurological disorders. For subjects that are genotyped or determined to be either APOE reference or heterozygous for an APOE predicted loss-of-function variant, such subjects can be treated with an APOE inhibitor.

In any of the embodiments described herein, the APOE predicted loss-of-function variant nucleic acid molecule can be any APOE nucleic acid molecule (such as, for example, genomic nucleic acid molecule, mRNA molecule, or cDNA molecule) resulting in a partial loss-of-function, a complete loss-of-function, a predicted partial loss-of-function, or a predicted complete loss-of-function of APOE activity. For example, the APOE predicted loss-of-function variant nucleic acid molecule is an APOE genomic nucleic acid molecule having an adjacent non-coding region comprising the SNP rs1065853.

In any of the embodiments described herein, the age-related diseases comprise cardiovascular disease, diabetes, atherosclerosis, obesity, cancer, infection, immunosenescence, coronary artery disease, and neurological disorders. In any of the embodiments described herein, the age-related disease is cardiovascular disease. In any of the embodiments described herein, the age-related disease is diabetes. In any of the embodiments described herein, the age-related disease is atherosclerosis. In any of the embodiments described herein, the age-related disease is immunosenescence. In any of the embodiments described herein, the age-related disease is obesity. In any of the embodiments described herein, the age-related disease is cancer. In any of the embodiments described herein, the age-related disease is infection. In any of the embodiments described herein, the age-related disease is coronary artery disease. In any of the embodiments described herein, the age-related disease is a neurological disorder, such as Alzheimer's disease.

As used herein, the term “longevity” refers to the lifespan of an organism. For example, a human having a lifespan of 100 years is considered to have increased longevity compared to a human having a lifespan of less than 100 years (e.g., 90 years, 80 years, 70 years, or 60 years, or less). Although lifespan can be measured in an individual organism, it is common to measure and compare mean or median lifespan of populations of individual organisms. In addition, the lifespan of a group of individuals that are APOE reference is expected to be shorter than the lifespan of a group of individuals that are heterozygous or homozygous for an APOE predicted loss-of-function variant nucleic acid molecule. Any inhibition or delay of development or severity of any of the age-related diseases and/or any increase in longevity is considered to be an inducement of healthy aging.

The present disclosure provides methods of increasing longevity in a subject, the methods comprising administering an APOE inhibitor to the subject.

The present disclosure also provides methods of inducing healthy aging in a subject, the methods comprising administering an APOE inhibitor to the subject.

In some embodiments, the APOE inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an APOE mRNA. In some embodiments, the antisense RNA, siRNA, or shRNA hybridizes to a sequence within an APOE genomic nucleic acid molecule or mRNA molecule and decreases expression of the APOE polypeptide in a cell in the subject. In some embodiments, the APOE inhibitor comprises an antisense RNA that hybridizes to an APOE genomic nucleic acid molecule or mRNA molecule and decreases expression of the APOE polypeptide in a cell in the subject. In some embodiments, the APOE inhibitor comprises an siRNA that hybridizes to an APOE genomic nucleic acid molecule or mRNA molecule and decreases expression of the APOE polypeptide in a cell in the subject. In some embodiments, the APOE inhibitor comprises an shRNA that hybridizes to an APOE genomic nucleic acid molecule or mRNA molecule and decreases expression of the APOE polypeptide in a cell in the subject.

In some embodiments, the APOE inhibitor comprises a nuclease agent that induces one or more nicks or double-strand breaks at a recognition sequence(s) or a DNA-binding protein that binds to a recognition sequence within an APOE genomic nucleic acid molecule. The recognition sequence can be located within a coding region of the APOE gene, or within regulatory regions (i.e., non-coding regions) that influence the expression of the gene. A recognition sequence of the DNA-binding protein or nuclease agent can be located in an intron, an exon, a promoter, an enhancer, a regulatory region, or any non-coding region. The recognition sequence can include or be proximate to the start codon of the APOE gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an APOE genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of APOE nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an APOE genomic nucleic acid molecule or it can be a nickase that creates a single-strand break in an APOE genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of APOE genomic nucleic acid molecules (including adjacent non-coding regions) can be generated by contacting a cell with a Cas protein and one or more gRNAs that hybridize to one or more gRNA recognition sequences within a target genomic locus or non-coding locus in the APOE genomic nucleic acid molecule. For example, a gRNA recognition sequence can be located within a region of SEQ ID NO:1 (i.e., non-coding region adjacent to the APOE gene). The gRNA recognition sequence can also include or be proximate to a position corresponding to position 501 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 501 according to SEQ ID NO:1. The gRNA recognition sequence can include or be proximate to the start codon of an APOE genomic nucleic acid molecule or the stop codon of an APOE genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an APOE genomic nucleic acid molecule or a non-coding locus adjacent to an APOE genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an APOE genomic nucleic acid molecule or non-coding region adjacent thereto. An exemplary gRNA is a gRNA effective to direct a Cas enzyme to bind to or cleave an APOE genomic nucleic acid molecule or non-coding region adjacent thereto, wherein the gRNA comprises a DNA-targeting segment that hybridizes to a gRNA recognition sequence within the APOE genomic nucleic acid molecule or non-coding region adjacent thereto that includes or is proximate to a position corresponding to position 501 according to SEQ ID NO:1. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of a position corresponding to position 501 according to SEQ ID NO:1. Other exemplary gRNAs comprise a DNA-targeting segment that hybridizes to a gRNA recognition sequence present within an APOE genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human APOE reference gene (or non-coding region adjacent thereto) are set forth in Table 1 as SEQ ID NOs:22-39.

TABLE 1
Guide RNA Recognition Sequences
NearAPOE Variation(s)

| Strand | gRNA Recognition Sequence | SEQ ID NO: |
|---|---|---|
| + | GACTACAGGCGCATACCACT | 22 |
| + | GCATACCACTAGGATTAATT | 23 |
| − | CCCCCCCAAATTAATCCTAG | 24 |
| − | GCGCCTGTAGTCTCAGCTAC | 25 |
| + | ACCACTAGGATTAATTTGGG | 26 |
| − | ATCACTTATCACTTGAGTCC | 27 |
| + | TTTCCAGTAGCTGAGACTAC | 28 |
| + | CCACTAGGATTAATTTGGGG | 29 |
| − | ACTTATCACTTGAGTCCAGG | 30 |
| − | TGGGAGTATCGCTTGAGCCC | 31 |
| + | TACCACTAGGATTAATTTGG | 32 |
| − | CCAGGAATTCCACATCAGCC | 33 |
| + | CATACCACTAGGATTAATTT | 34 |
| − | TGTAGTCTCAGCTACTGGAA | 35 |
| + | CCAGGCTGATGTGGAATTCC | 36 |
| + | CACTAGGATTAATTTGGGGG | 37 |
| + | ATACCACTAGGATTAATTTG | 38 |
| + | GGGGTCTGGCTTTGTTGGCC | 39 |

The Cas protein and the gRNA form a complex, and the Cas protein cleaves the target APOE genomic nucleic acid molecule. The Cas protein can cleave the nucleic acid molecule at a site within or outside of the nucleic acid sequence present in the target APOE genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the APOE genomic nucleic acid molecule to which a DNA-targeting segment of a gRNA will bind.

Such methods can result, for example, in an APOE genomic nucleic acid molecule or non-coding region adjacent thereto in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the APOE genomic nucleic acid molecule or non-coding region adjacent thereto. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the APOE inhibitor comprises a small molecule. In some embodiments, the APOE inhibitor comprises a soluble receptor for LDL (LDLR), such as the recombinant human LDL R 2148-LD/CF (R&D Systems). In some embodiments, the APOE inhibitor comprises an antibody (or antigen-binding fragment thereof), such as AB947 (Millipore), NB 110-60531 (Novus Biologicals), LS-B6780/43356 (Lifespan Bioscience), and EP1373Y (Epitomics). In some embodiments, the APOE inhibitor is an APOE4-specific antibody such as those commercially available from Bio Vision, MBL International, Covance, and/or IBL (American Immuno-Biological Laboratories). Methods for identifying additional inhibitory small molecules are known in the art (see, for example, Wang et al., Cell, 2015, 160, 1061-71).

In some embodiments, the methods of treatment further comprise detecting the presence or absence of an APOE predicted loss-of-function variant nucleic acid molecule in a biological sample from the subject. As used throughout the present disclosure, an “APOE predicted loss-of-function variant nucleic acid molecule” is any APOE nucleic acid molecule (such as, for example, genomic nucleic acid molecule, mRNA molecule, or cDNA molecule) resulting in a partial loss-of-function, a complete loss-of-function, a predicted partial loss-of-function, or a predicted complete loss-of-function in APOE activity.

The present disclosure also provides methods of increasing longevity or inducing healthy aging in a subject, wherein the subject is at risk of decreased longevity and/or is suffering from one or more age-related diseases. The methods comprise determining whether the subject has an APOE) predicted loss-of-function variant nucleic acid molecule by obtaining or having obtained a biological sample from the subject, and performing or having performed a genotyping assay on the biological sample to determine if the subject has a genotype comprising the APOE predicted loss-of-function variant nucleic acid molecule. When the subject is APOE reference, then an APOE inhibitor is administered or continued to be administered to the subject in a standard dosage amount. When the subject is heterozygous for an APOE predicted loss-of-function variant, then an APOE inhibitor is administered or continued to be administered to the subject in an amount that is lower than a standard dosage amount. The presence of a genotype having the APOE predicted loss-of-function variant nucleic acid molecule indicates the subject has a reduced risk of lower longevity and/or developing one or more age-related diseases.

Detecting the presence or absence of an APOE predicted loss-of-function variant nucleic acid molecule in a biological sample from a subject and/or determining whether a subject has an APOE predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the dose of the APOE inhibitor can be reduced by about 10%, by about 20%, by about 30%, by about 40%, by about 50%, by about 60%, by about 70%, by about 80%, or by about 90% for subjects that are heterozygous for an APOE predicted loss-of-function variant (i.e., a lower than the standard dosage amount) compared to subjects that are APOE reference (who may receive a standard dosage amount). In some embodiments, the dose of the APOE inhibitor can be reduced by about 10%, by about 20%, by about 30%, by about 40%, or by about 50%. In addition, the dose of the APOE inhibitor administered to subjects that are heterozygous for an APOE predicted loss-of-function variant can be administered less frequently compared to subjects that are APOE reference.

Administration of the APOE inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the APOE inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in an age-related disease, a decrease/reduction in the severity of an age-related disease (such as, for example, a reduction or inhibition of development of an age-related disease), a decrease/reduction in symptoms and age-related diseases-related effects, delaying the onset of symptoms and age-related diseases-related effects, reducing the severity of symptoms of age-related diseases-related effects, reducing the severity of an acute episode, reducing the number of symptoms and age-related diseases-related effects, reducing the latency of symptoms and age-related diseases-related effects, an amelioration of symptoms and age-related diseases-related effects, reducing secondary symptoms, reducing secondary infections, preventing relapse to age-related diseases, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of one or more age-related diseases development/progression (such as, for example, a complete or partial avoidance/inhibition or a delay), and an increased survival time (i.e., longevity) of the affected host animal, following administration of a therapeutic protocol. Treatment of age-related diseases encompasses the treatment of subjects already diagnosed as having any form of one or more age-related diseases at any clinical stage or manifestation, the delay of the onset or evolution or aggravation or deterioration of the symptoms or signs of age-related diseases, and/or preventing and/or reducing the severity of age-related diseases.

The present disclosure also provides methods of identifying a subject having an increased risk for lower longevity and/or developing one or more age-related diseases. In some embodiments, the methods comprise determining or having determined in a biological sample obtained from the subject the presence or absence of an APOE predicted loss-of-function variant nucleic acid molecule (such as a genomic nucleic acid molecule, mRNA molecule, and/or cDNA molecule). When the subject lacks an APOE predicted loss-of-function variant nucleic acid molecule (i.e., the subject is genotypically categorized as an APOE reference), then the subject has an increased risk for lower longevity and/or developing one or more age-related diseases. When the subject has an APOE predicted loss-of-function variant nucleic acid molecule (i.e., the subject is heterozygous for an APOE predicted loss-of-function variant nucleic acid molecule or homozygous for an APOE predicted loss-of-function variant nucleic acid molecule), then the subject has a decreased risk for lower longevity and/or developing one or more age-related diseases.

Having a single copy of an APOE predicted loss-of-function variant nucleic acid molecule is more protective of a subject from having lower longevity and/or developing age-related diseases than having no copies of an APOE predicted loss-of-function variant nucleic acid molecule. Without intending to be limited to any particular theory or mechanism of action, it is believed that a single copy of an APOE predicted loss-of-function variant nucleic acid molecule (i.e., heterozygous for an APOE predicted loss-of-function variant) is protective of a subject from having lower longevity and/or developing age-related diseases, and it is also believed that having two copies of an APOE predicted loss-of-function variant nucleic acid molecule (i.e., homozygous for an APOE predicted loss-of-function variant) may be more protective of a subject from having lower longevity and/or developing age-related diseases, relative to a subject with a single copy. Thus, in some embodiments, a single copy of an APOE predicted loss-of-function variant nucleic acid molecule may not be completely protective, but instead, may be partially or incompletely protective of a subject from having lower longevity and/or developing age-related diseases. While not desiring to be bound by any particular theory, there may be additional factors or molecules involved in longevity and/or the development of age-related diseases that are still present in a subject having a single copy of an APOE predicted loss-of-function variant nucleic acid molecule, thus resulting in less than complete protection from having lower longevity and/or development of age-related diseases.

Determining whether a subject has an APOE predicted loss-of-function variant nucleic acid molecule in a biological sample from a subject and/or determining whether a subject has an APOE predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of lower longevity and/or developing one or more age-related diseases, the subject is further treated with an APOE inhibitor, as described herein. For example, when the subject is APOE reference, and therefore has an increased risk of lower longevity and/or developing one or more age-related diseases, the subject is administered an APOE inhibitor in a standard dosage amount. In some embodiments, such a subject is also administered a therapeutic agent that treats or inhibits one or more age-related diseases. In some embodiments, when the subject is heterozygous for an APOE predicted loss-of-function variant, the subject is administered an APOE inhibitor in a dosage amount that is lower than a standard dosage amount. In some embodiments, such a subject is also administered a therapeutic agent that treats or inhibits one or more age-related diseases, but in a dosage amount that is less than the dosage amount for an APOE reference subject. In some embodiments, the subject is heterozygous for an APOE predicted loss-of-function variant.

In some embodiments, the methods can further comprise determining the subject's aggregate burden of not having a predicted loss-of-function variant APOE genomic nucleic acid molecule associated with increased longevity and/or decreased risk of developing one or more age-related diseases, predicted loss-of-function variant APOE mRNA molecules associated with increased longevity and/or decreased risk of developing one or more age-related diseases, and/or predicted loss-of-function variant APOE cDNA molecules produced from the mRNA molecules associated with increased longevity and/or decreased risk of one or more age-related diseases. The aggregate burden is the sum of all rare variants in the APOE gene (or non-coding regions adjacent thereto), which can be carried out in an association analysis with longevity or one or more age-related diseases. In some embodiments, the subject is homozygous for one or more predicted loss-of-function variant APOE nucleic acid molecules associated with increased longevity and/or decreased risk of developing one or more age-related diseases. In some embodiments, the subject is heterozygous for one or more predicted loss-of-function variant APOE nucleic acid molecules associated with increased longevity and/or decreased risk of developing one or more age-related diseases. The result of the association analysis may suggest that rare loss-of-function and missense variants of APOE may be associated with increased longevity and decreased risk of one or more age-related diseases. In some embodiments, when a subject is identified as having an increased risk of lower longevity and/or of developing one or more age-related diseases based on their aggregate burden, the subject is treated with an APOE inhibitor, as described herein.

The present disclosure also provides methods of identifying a subject having an increased risk of lower longevity and/or of developing an age-related disease, such as cardiovascular disease, diabetes, atherosclerosis, obesity, cancer, infection, immunosenescence, and neurological disorders, wherein the methods comprise determining or having determined the subject's aggregate burden of not having one or more APOE predicted loss-of-function variant genomic nucleic acid molecules, one or more APOE predicted loss-of-function variant mRNA molecules, or one or more APOE predicted loss-of-function variant cDNA molecules. The greater the aggregate burden the subject has, the greater the risk for decreased longevity and/or developing one or more age-related diseases. The lower the aggregate burden the subject has, the lower the risk for lower longevity and/or one or more age-related diseases.

In some embodiments, the subject's aggregate burden of not having any one or more of APOE predicted loss-of-function variant nucleic acid molecules represents a weighted sum of not having a plurality of any of the predicted loss-of-function variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, or at least about 1,000 of genetic variants associated with increased longevity and/or decreased risk of having one or more age-related diseases. In some embodiments, when the subject has an aggregate burden of not having any one or more of APOE predicted loss-of-function variant nucleic acid molecules above a desired threshold score, the subject has an increased risk of lower longevity and/or developing one or more age-related diseases. In some embodiments, when the subject has an aggregate burden of not having any one or more of APOE predicted loss-of-function variant nucleic acid molecules below a desired threshold score, the subject has a decreased risk of lower longevity and/or developing one or more age-related diseases.

In some embodiments, the aggregate burden of not having any one or more of APOE predicted loss-of-function variant nucleic acid molecules may be divided into quintiles, e.g., top quintile, intermediate quintiles, and bottom quintile, wherein the top quintile of aggregate burden of not having any one or more of APOE predicted loss-of-function variant nucleic acid molecules corresponds to the highest risk group, and the bottom quintile of aggregate burden of not having any one or more of APOE predicted loss-of-function variant nucleic acid molecules corresponds to the lowest risk group. In some embodiments, a subject having a greater aggregate burden of not having any one or more of APOE predicted loss-of-function variant nucleic acid molecules comprise the highest weighted aggregate burdens, including, but not limited to the top 10%, top 20%, top 30%, top 40%, or top 50% of aggregate loss-of-function effect burdens from a subject population. In some embodiments, the identified genetic variant is rs1065853, having association with increased longevity and decreased risk of having one or more age-related diseases. In some embodiments, this may comprise variants of nucleotide sites in the vicinity of rs1065853 that have the same regulatory effects.

The present disclosure also provides methods of detecting the presence or absence of an APOE predicted loss-of-function variant genomic nucleic acid molecule in a biological sample from a subject, and/or an APOE predicted loss-of-function variant mRNA molecule in a biological sample from a subject, and/or an APOE predicted loss-of-function variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for APOE variant genomic nucleic acid molecules are only exemplary sequences. Other sequences for the APOE variant genomic nucleic acid molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any APOE variant nucleic acid molecule, preliminary processing designed to isolate or enrich the sample for the genomic DNA can be employed. A variety of techniques may be used for this purpose. When detecting the level of any APOE variant mRNA, different techniques can be used enrich the biological sample with mRNA. Various methods to detect the presence or level of an mRNA or the presence of a particular variant genomic DNA locus can be used.

In some embodiments, detecting a human APOE predicted loss-of-function variant nucleic acid molecule in a subject comprises assaying or genotyping a biological sample obtained from the subject to determine whether an APOE genomic nucleic acid molecule in the biological sample, and/or an APOE mRNA molecule in the biological sample, and/or an APOE cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an APOE predicted loss-of-function variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the nucleotide sequence comprises a thymine at a position corresponding to position 501 according to SEQ ID NO:2 (for genomic nucleic acid molecules). In some embodiments, the nucleotide sequence comprises a thymine at a position corresponding to position 501 according to SEQ ID NO:2, or the complement thereof.

In some embodiments, the biological sample comprises a cell or cell lysate. Such methods can further comprise, for example, obtaining a biological sample from the subject comprising an APOE genomic nucleic acid molecule or mRNA molecule, and if mRNA, optionally reverse transcribing the mRNA into cDNA. Such assays can comprise, for example determining the identity of these positions of the particular APOE nucleic acid molecule. In some embodiments, the method is an in vitro method.

In some embodiments, the determining step, detecting step, or genotyping assay comprises sequencing at least a portion of the nucleotide sequence of the APOE genomic nucleic acid molecule, the APOE mRNA molecule, or the APOE cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or genotyping assay comprises sequencing at least a portion of the nucleotide sequence of the APOE genomic nucleic acid molecule in the biological sample, wherein the sequenced portion comprises a position corresponding to position 501 according to SEQ ID NO:2, or the complement thereof. When the sequenced portion of the APOE nucleic acid molecule in the biological sample comprises a thymine at a position corresponding to position 501 according to SEQ ID NO:2, then the APOE nucleic acid molecule in the biological sample is an APOE predicted loss-of-function variant nucleic acid molecule.

In some embodiments, the determining step, detecting step, or genotyping assay comprises: a) contacting the biological sample with a primer hybridizing to a portion of the nucleotide sequence of the APOE genomic nucleic acid molecule that is proximate to a position corresponding to position 501 according to SEQ ID NO:2; b) extending the primer at least through the position of the nucleotide sequence of the APOE genomic nucleic acid molecule corresponding to position 501 according to SEQ ID NO:2; and c) determining whether the extension product of the primer comprises a thymine at a position corresponding to position 501 according to SEQ ID NO:2.

In some embodiments, the assay comprises sequencing the entire nucleic acid molecule. In some embodiments, only an APOE genomic nucleic acid molecule is analyzed. In some embodiments, only an APOE mRNA is analyzed. In some embodiments, only an APOE cDNA obtained from APOE mRNA is analyzed.

In some embodiments, the determining step, detecting step, or genotyping assay comprises: a) amplifying at least a portion of the APOE genomic nucleic acid molecule, wherein the amplified portion comprises a thymine at a position corresponding to position 501 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 501 according to SEQ ID NO:2, or the complement thereof; and d) detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 501 according to SEQ ID NO:2, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an APOE variant genomic sequence, variant mRNA sequence, or variant cDNA sequence and not the corresponding APOE reference sequence under stringent conditions, and determining whether hybridization has occurred.

In some embodiments, the methods utilize probes and primers of sufficient nucleotide length to bind to the target nucleotide sequence and specifically detect and/or identify a polynucleotide comprising an APOE variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an APOE genomic nucleic acid molecule, or complement thereof, within a biological sample comprises a nucleotide sequence comprising a thymine at a position corresponding to position 501 according to SEQ ID NO:2, the biological sample can be subjected to an amplification method using a primer pair that includes a first primer derived from the 5′ flanking sequence adjacent to a thymine at a position corresponding to position 501 according to SEQ ID NO:2 and a second primer derived from the 3′ flanking sequence adjacent to a thymine at a position corresponding to position 501 according to SEQ ID NO:2 to produce an amplicon that is indicative of the presence of the SNP at positions encoding a thymine at a position corresponding to position 501 according to SEQ ID NO:2. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 501 according to SEQ ID NO:2, and at least 1, 2, 3, 4, 5, 6, 7, 8, 9, 10, or more nucleotides on each side of positions comprising a thymine at a position corresponding to position 501 according to SEQ ID NO:2.

PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides isolated nucleic acid molecules that hybridize to APOE variant genomic nucleic acid molecules, APOE variant mRNA molecules, and/or APOE variant cDNA molecules. In some embodiments, the isolated nucleic acid molecules hybridize to a portion of the APOE genomic nucleic acid molecule that includes a position corresponding to position 501 according to SEQ ID NO:2.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to APOE variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to APOE variant genomic nucleic acid molecules, APOE variant mRNA molecules, and/or APOE variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of the APOE genomic nucleic acid molecule, wherein the portion comprises a position corresponding to position 501 according to SEQ ID NO:2, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the APOE variant genomic nucleic acid molecules disclosed herein. The primers described herein can be used to amplify APOE genomic variant genomic nucleic acid molecules, or a fragment thereof.

The present disclosure also provides pairs of primers comprising any of the primers described above. For example, if one of the primers' 3′-ends hybridizes to a guanine at a position corresponding to position 501 according to SEQ ID NO:1 (rather than thymine) in a particular APOE genomic nucleic acid molecule, then the presence of the amplified fragment would indicate the presence of an APOE reference genomic nucleic acid molecule. Conversely, if one of the primers' 3′-ends hybridizes to a thymine at a position corresponding to position 501 according to SEQ ID NO:2 (rather than guanine) in a particular APOE genomic nucleic acid molecule, then the presence of the amplified fragment would indicate the presence of the APOE variant genomic nucleic acid molecule. In some embodiments, the nucleotide of the primer complementary to the thymine at a position corresponding to position 501 according to SEQ ID NO:2 can be at the 3′ end of the primer.

In the context of the disclosure “specifically hybridizes” means that the probe or primer (such as, for example, the alteration-specific probe or alteration-specific primer) does not hybridize to a nucleic acid sequence encoding an APOE reference genomic nucleic acid molecule, an APOE reference mRNA molecule, and/or an APOE reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of an APOE reference genomic nucleic acid molecule is set forth in SEQ ID NO:3. The nucleotide sequence of a non-coding region adjacent to the APOE reference genomic nucleic acid molecule is set forth in SEQ ID NO:1. Referring to SEQ ID NO:1, position 501 is a guanine.

The nucleotide sequence of the same non-coding region adjacent to the APOE reference genomic nucleic acid molecule but comprising the rs1065853 SNP is set forth in SEQ ID NO:2. Referring to SEQ ID NO:2, position 501 is a thymine.

The nucleotide sequence of an APOE reference mRNA molecule is set forth in SEQ ID NO:4. The nucleotide sequence of another APOE reference mRNA molecule is set forth in SEQ ID NO:5. The nucleotide sequence of another APOE reference mRNA molecule is set forth in SEQ ID NO:6. The nucleotide sequence of another APOE reference mRNA molecule is set forth in SEQ ID NO:7. The nucleotide sequence of another APOE reference mRNA molecule is set forth in SEQ ID NO:8. The nucleotide sequence of another APOE reference mRNA molecule is set forth in SEQ ID NO:9. The nucleotide sequence of another APOE reference mRNA molecule is set forth in SEQ ID NO:10. The nucleotide sequence of another APOE reference mRNA molecule is set forth in SEQ ID NO:11.

The nucleotide sequence of an APOE reference cDNA molecule is set forth in SEQ ID NO:12. The nucleotide sequence of another APOE reference cDNA molecule is set forth in SEQ ID NO:13. The nucleotide sequence of another APOE reference cDNA molecule is set forth in SEQ ID NO:14. The nucleotide sequence of another APOE reference cDNA molecule is set forth in SEQ ID NO:15. The nucleotide sequence of another APOE reference cDNA molecule is set forth in SEQ ID NO:16. The nucleotide sequence of another APOE reference cDNA molecule is set forth in SEQ ID NO:17. The nucleotide sequence of another APOE reference cDNA molecule is set forth in SEQ ID NO:18. The nucleotide sequence of another APOE reference cDNA molecule is set forth in SEQ ID NO:19.

The amino acid sequence of an APOE reference polypeptide (NP_000032.1) is set forth in SEQ ID NO:20, and is 317 amino acids in length. The amino acid sequence of another APOE reference polypeptide (NP_001289617.1) is set forth in SEQ ID NO:21, and is 343 amino acids in length.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, an APOE genomic nucleic acid molecule comprising a nucleotide sequence that comprises a thymine at a position corresponding to position 501 according to SEQ ID NO:2 means that if the nucleotide sequence of the APOE genomic nucleic acid molecule is aligned to the sequence of SEQ ID NO:2, the APOE sequence has a thymine residue at the position that corresponds to position 501 of SEQ ID NO:2. Herein, such a sequence is also referred to as an APOE rs1065853 sequence referring to genomic nucleic acid molecules.

As described herein, a position within an APOE genomic nucleic acid molecule that corresponds to position 501 according to SEQ ID NO:2, for example, can be identified by performing a sequence alignment between the nucleotide sequence of a particular APOE nucleic acid molecule and the nucleotide sequence of SEQ ID NO:2. A variety of computational algorithms exist that can be used for performing a sequence alignment to identify a nucleotide position that corresponds to, for example, position 501 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The present disclosure also provides APOE inhibitors for use in increasing longevity and/or inducing healthy aging in a subject having an APOE genomic nucleic acid molecule having a nucleotide sequence comprising a thymine at a position corresponding to position 501 according to SEQ ID NO:2, or the complement thereof.

In some embodiments, the APOE inhibitor is an antisense nucleic acid molecule, a small interfering RNA (siRNA), or a short hairpin RNA (shRNA) that hybridizes to an APOE mRNA.

In some embodiments, the APOE inhibitor comprises a Cas protein and guide RNA (gRNA) that hybridizes to a gRNA recognition sequence within an APOE genomic nucleic acid molecule. In some embodiments, the Cas protein is Cas9 or Cpf1. In some embodiments, the gRNA recognition sequence includes or is proximate to position 501 according to SEQ ID NO:1. In some embodiments, the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 501 according to SEQ ID NO:1. In some embodiments, a Protospacer Adjacent Motif (PAM) sequence is about 2 to about 6 nucleotides downstream of the gRNA recognition sequence. In some embodiments, the gRNA comprises from about 17 to about 23 nucleotides. In some embodiments, the gRNA recognition sequence comprises a nucleotide sequence according to any one of SEQ ID NOs:22-39.

In some embodiments, the APOE inhibitor comprises a soluble receptor for LDL (LDLR). In some embodiments, the APOE inhibitor comprises an antibody.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Databases

The UK Biobank study (UKB) has been described in detail previously (Bycroft et al., Nature, 2018, 562, 203-209). The UKB recruited about 500,000 participants from the UK between 2006 and 2010. Participants attended one of the 22 recruitment centers across the UK where they provided a blood sample for DNA extraction and biomarker analysis, and completed questionnaires covering a wide range of medical, social, and lifestyle information. Information on biomarker levels was retrieved from data fields 30882 Urate, 30712 C-reactive protein, 30722 Cystatin-C, 30762 HDL-cholesterol, 30782 LDL-direct, and 30872 Triglycerides. Biomarker raw values were residualized for top ten principal components, sex, age, age², and Age-x-Sex, and rank inverse normal transformed. The version three of the imputed genotype data was used. A description of data can be found at the world wide web (see, “biobank.ctsu.ox.ac.uk/crystal/label.cgi?id=263). There are 805,426 variants in the released genotype data. Genotype data were quality controlled, phased and about 96M genotypes were imputed using the Haplotype Reference Consortium and UK10K haplotype resources. Association analysis of APOE genotypes with the six quantitative phenotypes was performed with the lm( ) function contained in the R statistical analysis software.

### Example 2: A Common APOE Noncoding Variant has Effects on Biomarker Traits that are Independent from the APOE e4 Variant

Raw measurements including UA, CYSC, CRP, HDL, LDL, and TRIG were residualized for the top ten ancestry PCs as well as for the potential modifiers sex and age. To bring effects for different traits on a same scale, residuals were rank inverse normal transformed. In the core interval from 44.75 Mb to 45 Mb around the APOE gene on chromosome 19, the analysis included 864 variants with minor allele frequency above 1% and less than 5% missing hard call genotypes among 337,484 unrelated European individuals.

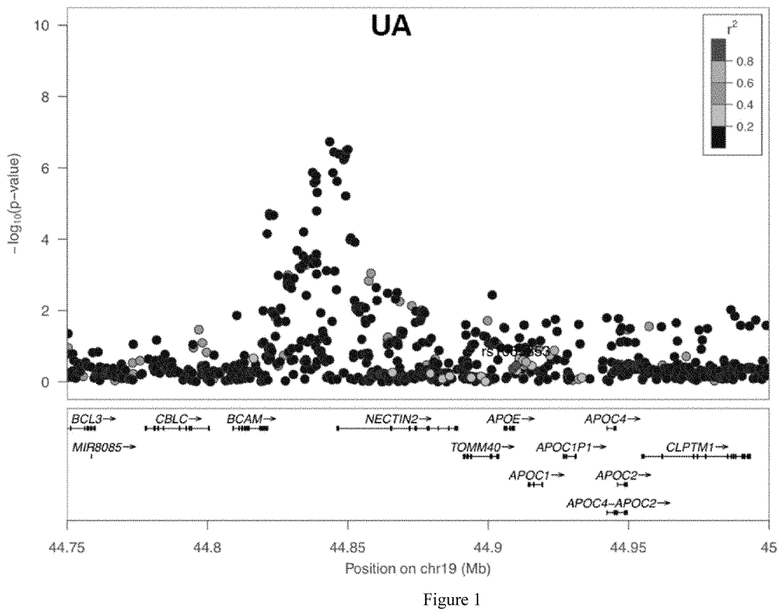

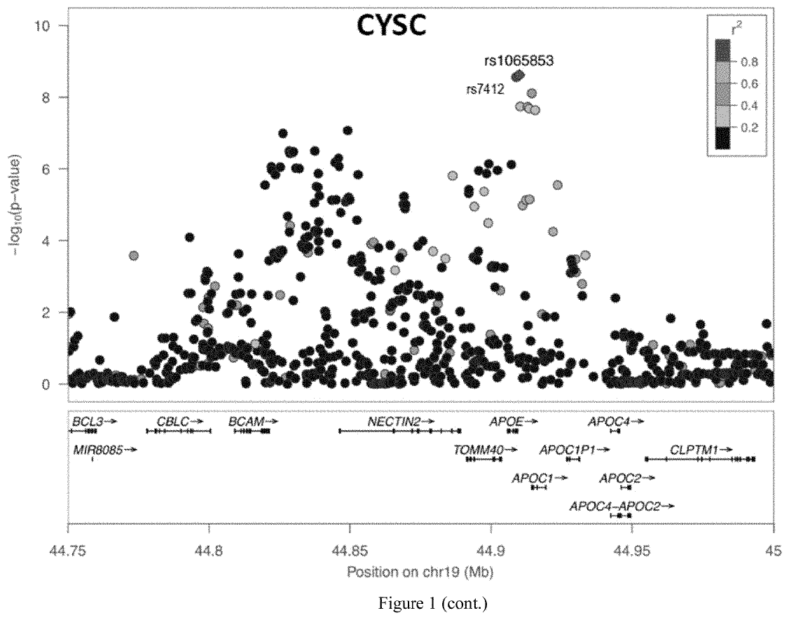

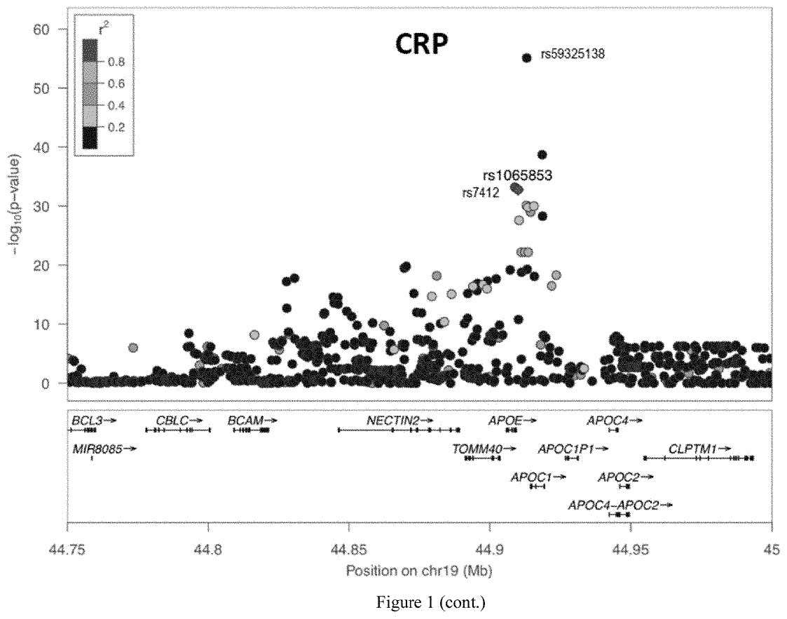

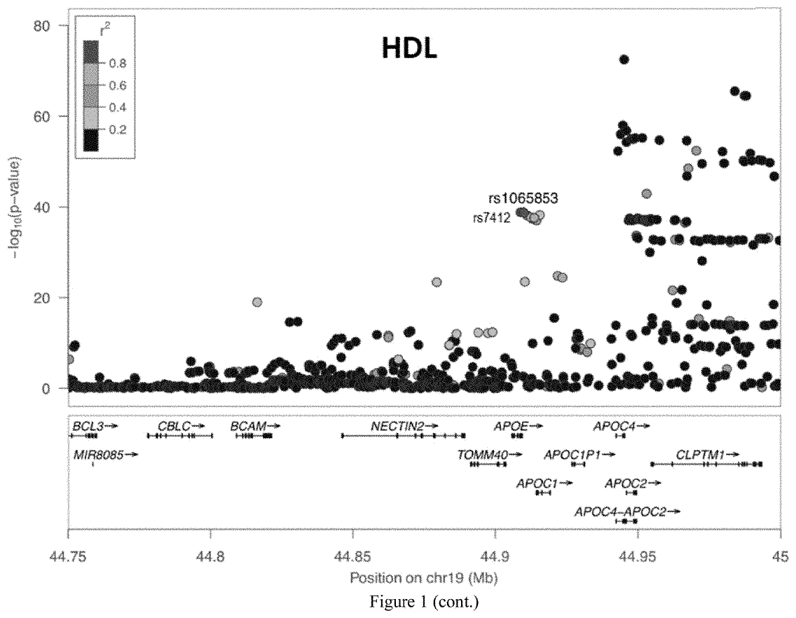

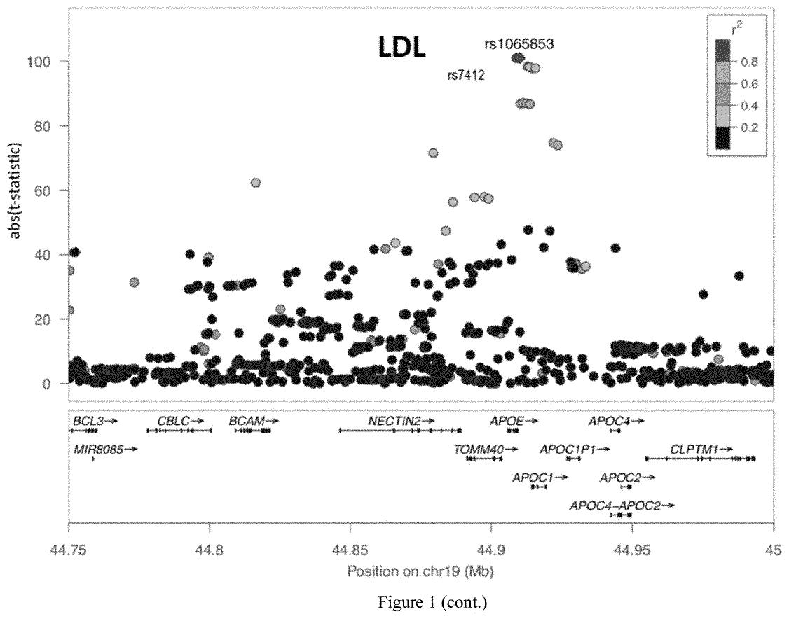

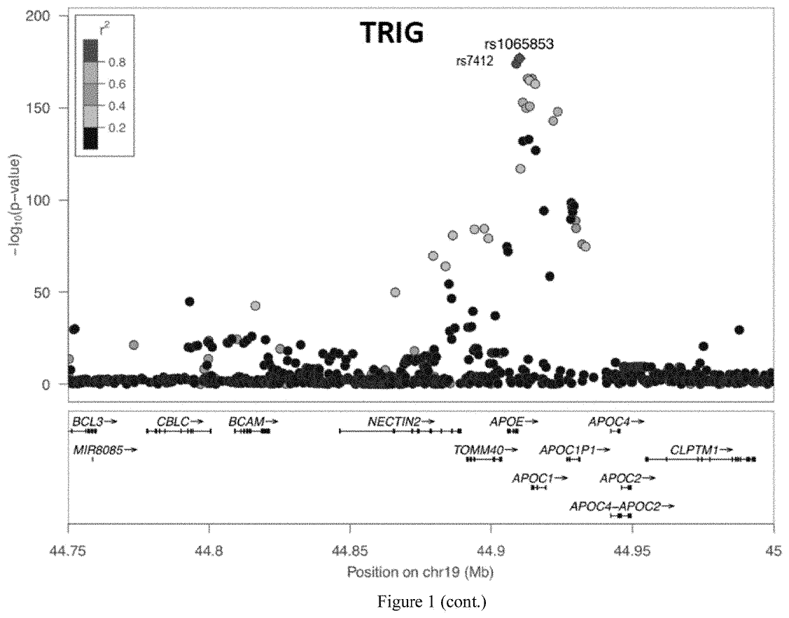

These variants were tested for their association with the above six traits while conditioning on rs429358 (see, FIG. 1), which marks the APOE e4 haplotype. FIG. 1 shows the association of the 864 variants located in the interval from 44.75 Mb to 45 Mb around the APOE gene on chromosome 19 with UA, CYSC, CRP, HDL, LDL, and TRIG traits.

When conditioning on rs429358, the variant rs1065853 (19:44909976:G:T) displayed the strongest association with CYSC (beta=0.027, P<1e-8), HDL (beta=0.063, P <1e-38), LDL (beta=−0.46, P<1e-325), and TRIG (beta=0.13, P<1e-176). The variant rs1065853 also showed an association with CRP (beta=0.0547, P<1e-32) and has little LD to the CRP lead variant of this analysis step (r559325138, 19:44913034:C:T), indicating independent effects of the two variants. There is no significant association with UA for rs1065853 (P >0.05) despite the known association of the APOE e4 haplotype with UA.

The evaluation of rs429358 together with rs1065853 in a joint model (see, Table 2) showed two statistically independent effects for the two variants. Table 2 shows association of rs429358 and rs1065853 with UA, CYSC, CRP, HDL, LDL, and TRIG in a joint model.

TABLE 2

| Phenotype | Variant | Beta | CI95 | Pval |
|---|---|---|---|---|
| UA | rs429358 | −0.0202 | (−0.027, −0.013) | 4.86E−09 |
| | rs1065853 | −0.0043 | (−0.013, 0.0048) | 0.352 |
| CYSC | rs429358 | −0.0323 | (−0.039, −0.026) | 8.49E−21 |
| | rs1065853 | 0.0275 | (0.018, 0.037) | 2.40E−09 |
| CRP | rs429358 | −0.238 | (−0.25, −0.23) | <E−325 |
| | rs1065853 | 0.0547 | (0.046, 0.064) | 1.44E−33 |
| HDL | rs429358 | −0.0745 | (−0.082, −0.067) | 4.87E−94 |
| | rs1065853 | 0.0634 | (0.054, 0.073) | 1.80E−39 |
| LDL | rs429358 | 0.153 | (0.15, 0.16) | <E−325 |
| | rs1065853 | −0.457 | (−0.47, −0.45) | <E−325 |
| TRIG | rs429358 | 0.0764 | (0.07, 0.083) | 3.17E−108 |
| | rs1065853 | 0.131 | (0.12, 0.14) | 1.18E−177 |

| Phenotype | Variant | N Ref:Het:Alt | Raw Mean Ref:Het:Alt |
|---|---|---|---|
| UA | rs429358 | 228664:84833:7846 | 309.88, 308.75, 306.31 |
| | rs1065853 | 271349:47565:2078 | 309.51, 309.42, 308.39 |
| CYSC | rs429358 | 228941:84939:7856 | 0.91052, 0.90478, 0.9031 |
| | rs1065853 | 271670:47633:2082 | 0.9082, 0.01186, 0.9205 |
| CRP | rs429358 | 228467:84766:7827 | 2.7696, 2.1761, 1.6986 |
| | rs1065853 | 271100:47535:2075 | 2.5493, 2.7864, 2.876 |
| HDL | rs429358 | 209584:77800:7156 | 1.4612, 1.4318, 1.413 |
| | rs1065853 | 248549:43734:1932 | 1.4473, 1.4796, 1.4719 |
| LDL | rs429358 | 228520:84790:7840 | 3.5163, 3.693, 3.8124 |
| | rs1065853 | 271180:47544:2076 | 3.634, 3.2453, 2.6993 |
| TRIG | rs429358 | 228769:84874:7850 | 1.735, 1.8006, 1.8667 |
| | rs1065853 | 271472:47588:2082 | 1.738, 1.8374, 2.1717 |

Raw value for UA is given in um/I, for CYSC and CRP in mg/l, and for HDL, LDL, and TRIG in mmol/l. There exists considerable heterogeneity of the magnitude of e4 versus e2 effects across the six traits and the analysis shows that the two variants act independently from each other on the studied traits.

### Example 3: A Common APOE Noncoding Variant Causes the APOE e2 Effects that are Previously Attributed Only to the Arg176Cys Missense Variant

Nearly equal strong effects as for rs1065853 were present for the missense variant rs7412 (APOE Arg176Cys, 19:44908822:C:T), which is typically used in the literature to define the e2 haplotype. The location of rs1065853 is approximately 1.1 kb downstream of rs7412 and the two variants are in near perfect LD (r²=0.99). Thus, the two variants are mostly present together and can be exchangeably used to define the e2 haplotype.

Due to their high collinearity, the effects of rs1065853 and rs7412 cannot be evaluated and compared by fitting a joint model. Therefore, individuals were instead grouped whether they are homozygous reference at both sites (n=270,976), heterozygous at both sites (n=47,491), heterozygous at rs7412 and homozygous reference at rs1065853 (n=204), or homozygous reference at rs7412 and heterozygous at rs1065853 (n=46). Double heterozygous individuals showed the expected e2 effects as compared to the reference, whereas neither of the single heterozygote groups differed significantly from the reference for any of the six traits (data not shown). Because APOE e2 exerts fairly strong effect on LDL and TRIG, this absence of any significant difference cannot be easily explained by limited statistical power due to the small number of single heterozygotes. Rather, it gives rise to a model where double heterozygous but not single heterozygous for rs1065853 and rs7412 may differ from the reference as expected for the e2 haplotype. When this model was examined by comparing double heterozygotes at both sites to single heterozygotes at either site (see, Table 3), the latter have a significantly higher LDL (beta=0.31, P<1e-6) and lower TRIG (beta=−0.18, P<1e-2). Table 3 shows a comparison of double heterozygotes with single heterozygotes at rs1065853 and rs7412 LDL, and TRIG, which are the two traits with the strongest effects in above Example 2.

TABLE 3

| | Genotype | | | | | Raw | |
|---|---|---|---|---|---|---|---|
| | rs7412- | | | | | Measurement | Raw Standard |
| Trait | rs1065853 | N | Beta | Stde | Pval | Mean Value | Error of Mean |
| LDL | HH | 47491 | | | | 3.2451 | 0.0036 |
| | HR or RH | 250 | 0.306 | 0.0596 | 2.8e−7 | 3.5436 | 0.0508 |
| TRIG | HH | 47491 | | | | 1.8374 | 0.005 |
| | HR or RH | 250 | −0.181 | 0.0639 | 0.0047 | 1.6391 | 0.0643 |

Individuals heterozygous at both sites are denoted H—H, whereas individuals heterozygous at one site and homozygous reference are denoted HR or RH. Single heterozygotes have significantly higher LDL and lower TRIG and are, thus, significantly less “e2-like”. The differences between double and single heterozygotes for the other traits are directionally consistent, but do not reach significance. Thus, single heterozygotes are significantly different from double heterozygotes, but do not differ from the reference, e.g. from e3 homozygotes. This indicates that rs7412 and rs1065853 cannot be interpreted independently from each other and that rs1065853 has an important functional role for the e2 haplotype. It is assumed that rs1065853 exerts a loss-of-function effect on the APOE gene via a regulatory mechanism. Thus, mimicking this loss-of-function effect can be used for the beneficial health effects which are hitherto ascribed to rs7412.

### Example 4: Several Neighboring Variants Influence Biomarkers Independent from APOE Missense Variants

The Finemap algorithm (Benner et al., Bioinformatics, 2016, 32, 1493-1501) was applied to the summary statistics obtained by conditioning on both e4 (as tagged by r5429358) and e2 (as tagged by r57412). To be maximally comprehensive, all variants within the larger interval from 1.5 Mb upstream to 1.5 Mb downstream of the APOE gene were included. For all six traits this analysis provided evidence for additional causal variants that are independent from the two missense variants, although the number of such variants and the level of confidence varies across traits (Table 4). Three high confidence causal variant predictions (posterior probability >0.95) that are located in the above defined core interval (44.75-45 Mb) were recognized. Two of them are predicted causal across three traits each.

TABLE 4

| | N causal posterior | Top causal variant predictions | |
|---|---|---|---|
| Trait | probability | in Core Interval (PP) | |
| CRP | 8 (0.09); 9 (0.6); | rs5112 (0.998); | rs35136575 (0.998) |
| | 10 (0.31) | | |
| HDL | 6 (0.06); 7 (0.87); | rs35136575 (1); | rs5167 (1) |
| | 8 (0.06) | | |
| LDL | 6 (0.13); 7 (0.7); | rs5112 (0.991); | rs35136575 (0.991); |
| | 8 (0.17) | | |
| TRIG | 5 (0.22); 6 (0.76) | rs5112 (0.986); | rs1064725 (0.969) |

Posterior probabilities (PP) for the number of causal variants at the APOE locus with effects independent from APOE e4 and e2 as estimated by the Finemap algorithm. Variants shown are predicted causal with high confidence (PP >0.95) by Finemap and falling in the core interval (44.75 Mb-45 Mb) on chromosome 19. The variant rs5112 (19:44927023:C:G) lowers CRP (beta=−0.047, P<1e-107), but increases LDL (beta=0.056, P<1e-16) and TRIG (beta=0.065, P<1e-16). The variant rs35136575 (19:44935906:C:G) increases CRP (beta=0.041, P <1e-16) and HDL (beta=0.037, P<1e-16), but lowers LDL (beta=−0.065, P<1e-113). Both variants are located downstream of APOE between the neighboring APOC4 and APOC1 genes. Furthermore, rs5167 (19:44945208:T:G), a Leu96Arg change in APOC4, is predicted causal for HDL (beta=0.048, P<1e-16) and rs1064725 is predicted causal for TRIG (19:44919304:T:G, beta=−0.029, P<1e-4).

### Example 5: Associations

Associations of heterogenous magnitude and direction were observed for rs429358 (Cys130Arg, known as e4) and rs7412 (Arg176Cys, known as e2) with the quantitative biomarkers UA, CYSC, CRP, HDL, LDL, and TRIG. These associations can be easily explained by coding variant effects only, because such variants would be expected to act more homogenously across different traits. The observation that rs7412 is in near perfect LD with rs1065853 raises the possibility that rs1065853 exerts a regulatory contribution to any e2 haplotype effects that are hitherto entirely attributed to the Arg176Cys (r57412) variant. In fact, it was found that individuals who are heterozygous either at rs7412 or rs1065853 are less “e2-like” than individuals who are heterozygous at both sites. This suggests that any understanding of the APOE e2 haplotype remains incomplete without a functional characterization of rs1065853.

The hypothesis that rs1065853 may act as a causal variant on the e2 haplotype could also explain the limited insights gained so far from modeling of APOE e2 as defined by Arg176Cys only. In fact, APOE e2 was even termed “the forgotten APOE allele”. This analysis raises the possibility that e2 remained enigmatic in its function, because Arg176Cys is not a primary causal variant driving the effects of the e2 haplotype. From the therapeutic perspective, this implies that therapeutic efforts to modify APOE activity should consider the potential alteration of APOE dosage by rs1065853.

From a theoretical perspective, the strong effects of APOE on health, and also longevity are surprising, given that most heritability is spread out homogeneously across the genome. Thus, from the general understanding of the architecture of complex diseases, it is highly unusual to observe effects of the magnitude of APOE. The resolution to this paradox comes from models indicating that genes with greater effects affect multiple traits in opposite direction. The observation herein of heterogenous effects across biomarker traits demonstrates that this explanation applies to APOE variants and the proposed regulatory effects of rs1065853 are accounting for the heterogeneity of effect sizes across traits.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of increasing longevity or inducing healthy aging in a subject, the method comprising administering an apolipoprotein E (APOE) inhibitor to the subject.

2. (canceled)

3. The method according to claim 1, wherein the APOE inhibitor comprises an antisense nucleic acid molecule, a small interfering RNA (siRNA), or a short hairpin RNA (shRNA) that hybridizes to an APOE mRNA.

4. The method according to claim 1, wherein the APOE inhibitor comprises a Cas protein and guide RNA (gRNA) that hybridizes to a gRNA recognition sequence within an APOE genomic nucleic acid molecule.

5-10. (canceled)

11. The method according to claim 1, wherein the APOE inhibitor comprises a soluble receptor for LDL (LDLR).

12. The method according to claim 1, wherein the APOE inhibitor comprises an antibody.

13. The method according to claim 1, further comprising detecting the presence or absence of an APOE predicted loss-of-function variant nucleic acid molecule in a biological sample from the subject.

14. The method according to claim 13, wherein when the subject is APOE reference, the subject is administered the APOE inhibitor in a standard dosage amount.

15. The method according to claim 13, wherein when the subject is heterozygous for an APOE predicted loss-of-function variant, the subject is administered the APOE inhibitor in a dosage amount that is lower than a standard dosage amount.

16. The method according to claim 13, wherein the APOE predicted loss-of-function variant nucleic acid molecule is a genomic nucleic acid molecule having a nucleotide sequence comprising a thymine at a position corresponding to position 501 according to SEQ ID NO:2.

17-22. (canceled)

23. A method of increasing longevity or inducing healthy aging in a subject, wherein the subject is at risk of decreased longevity and/or is suffering from one or more age-related diseases, the method comprising the steps of:
determining whether the subject has an apolipoprotein E (APOE) predicted loss-of-function variant nucleic acid molecule by:
obtaining or having obtained a biological sample from the subject; and
performing or having performed a genotyping assay on the biological sample to determine if the subject has a genotype comprising the APOE predicted loss-of-function variant nucleic acid molecule; and
when the subject is APOE reference, then administering or continuing to administer to the subject an APOE inhibitor in a standard dosage amount; and
when the subject is heterozygous for an APOE predicted loss-of-function variant, then administering or continuing to administer to the subject the APOE inhibitor in an amount that is lower than a standard dosage amount;
wherein the presence of a genotype having the APOE predicted loss-of-function variant nucleic acid molecule indicates the subject has a reduced risk of lower longevity and/or developing one or more age-related diseases.

24. The method according to claim 23, wherein the subject is APOE reference, and the subject is administered or continued to be administered the APOE inhibitor in a standard dosage amount.

25. The method according to claim 23, wherein the subject is heterozygous for an APOE predicted loss-of-function variant, and the subject is administered or continued to be administered the APOE inhibitor in an amount that is lower than a standard dosage amount.

26. The method according to claim 23, wherein the APOE predicted loss-of-function variant nucleic acid molecule is a genomic nucleic acid molecule having a nucleotide sequence comprising a thymine at a position corresponding to position 501 according to SEQ ID NO:2.

27. The method according to claim 23, wherein the genotyping assay comprises sequencing at least a portion of the nucleotide sequence of the APOE genomic nucleic acid molecule in the biological sample, wherein the sequenced portion comprises a position corresponding to position 501 according to SEQ ID NO:2, or the complement thereof;
wherein when the sequenced portion of the APOE genomic nucleic acid molecule in the biological sample comprises a thymine at a position corresponding to position 501 according to SEQ ID NO:2, then the APOE genomic nucleic acid molecule in the biological sample is an APOE predicted loss-of-function variant genomic nucleic acid molecule.

28. The method according to claim 23, wherein the genotyping assay comprises:
a) contacting the biological sample with a primer hybridizing to a portion of the nucleotide sequence of the APOE genomic nucleic acid molecule that is proximate to a position corresponding to position 501 according to SEQ ID NO:2;
b) extending the primer at least through the position of the nucleotide sequence of the APOE genomic nucleic acid molecule corresponding to position 501 according to SEQ ID NO:2; and
c) determining whether the extension product of the primer comprises a thymine at a position corresponding to position 501 according to SEQ ID NO:2.

29. (canceled)

30. The method according to claim 23, wherein the genotyping assay comprises:
a) amplifying at least a portion of the APOE genomic nucleic acid molecule, wherein the portion comprises a thymine at a position corresponding to position 501 according to SEQ ID NO:2, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 501 according to SEQ ID NO:2; and
d) detecting the detectable label.

31. The method according to claim 23, wherein the genotyping assay comprises:
contacting the APOE genomic nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 501 according to SEQ ID NO:2, or the complement thereof; and
detecting the detectable label.

32. (canceled)

33. The method according to claim 23, wherein the APOE inhibitor comprises an antisense nucleic acid molecule, a small interfering RNA (siRNA), or a short hairpin RNA (shRNA) that hybridizes to an APOE mRNA.

34. The method according to claim 23, wherein the APOE inhibitor comprises a Cas protein and guide RNA (gRNA) that hybridizes to a gRNA recognition sequence within an APOE genomic nucleic acid molecule.

35-40. (canceled)

41. The method according to claim 23, wherein the APOE inhibitor comprises a soluble receptor for LDL (LDLR).

42. The method according to claim 23, wherein the APOE inhibitor comprises an antibody.

43. A method of identifying a subject having an increased risk for lower longevity and/or developing one or more age-related diseases, wherein the method comprises:
determining or having determined the presence or absence of an apolipoprotein E (APOE) predicted loss-of-function variant nucleic acid molecule in a biological sample obtained from the subject;
wherein:
when the subject is APOE reference, then the subject has an increased risk for lower longevity and/or developing one or more age-related diseases; and
when the subject is heterozygous for an APOE predicted loss-of-function variant nucleic acid molecule or homozygous for an APOE predicted loss-of-function variant nucleic acid molecule, then the subject has a decreased risk for lower longevity and/or developing one or more age-related diseases.

44-73. (canceled)


SEQUENCE LISTING (39 sequences)

SEQ ID NO: 1 - DNA, 1001 bp, homo sapien
gccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttt

SEQ ID NO: 2 - DNA, 1001 bp, homo sapien
gccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttggggggggtggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttt

SEQ ID NO: 3 - DNA, 3598 bp, homo sapien
ctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 4 - RNA, 1166 nt, homo sapien
cuacucagccccagcggaggugaaggacguccuuccccaggagccgacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 5 - RNA, 1181 nt, homo sapien
cuacucagccccagcggaggugaaggacguccuuccccaggagccguuucuccuuccccagacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 6 - RNA, 1248 nt, homo sapien
cuacucagccccagcggaggugaaggacguccuuccccaggagccggugagaagcgcagucgggggcacggggaugagcucaggggccucuagaaagagcugggacccugggaaccccuggccuccagacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 7 - RNA, 1144 nt, homo sapien
agagacgacccgacccgcuagaagacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 8 - RNA, 1265 nt, homo sapien
ggauggggagauaagagaagaccaggagggaguuaaauagggaauggguugggggcggcuugguaaaugugcugggauuaggcuguugcagauaaugcaacaaggcuuggaaggcuaaccugggacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgcaucaaaaaaaaaaaaaaaaaaa

SEQ ID NO: 9 - RNA, 1208 nt, homo sapien
agggggagcccuauaauuggacaagucugggauccuugaguccuacucagccccagcggaggugaaggacguccuuccccaggagccgacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 10 - RNA, 1157 nt, homo sapien
ccccagcggaggugaaggacguccuuccccaggagccgacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaagucacccaagaacugagggcgcugauggacgagaccaugaaggaguugaaggccuacaaaucggaacuggaggaacaacugaccccgguagcggaggagacgcgggcacggcuguccaaggagcugcagacggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaucccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagucggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgc

SEQ ID NO: 11 - RNA, 1186 nt, homo sapien
ggacguccuuccccaggagccgacuggccaaucacaggcaggaagaugaagguucugugggcugcguugcuggucacauuccuggcaggaugccaggccaagguggagcaagcgguggagacagagccggagcccgagcugcgccagcagaccgaguggcagagcggccagcgcugggaacuggcacugggucgcuuuugggauuaccugcgcugggugcagacacugucugagcaggugcaggaggagcugcucagcucccaggucacccaggaacugagggcgcugauggacgagaccaugaaggaguugaaggccuacaaaucggaacuggaggaacaacugaccccgguggcggaggagacgcgggcacggcuguccaaggagcugcaggcggcgcaggcccggcugggcgcggacauggaggacgugugcggccgccuggugcaguaccgcggcgaggugcaggccaugcucggccagagcaccgaggagcugcgggugcgccucgccucccaccugcgcaagcugcguaagcggcuccuccgcgaugccgaugaccugcagaagcgccuggcaguguaccaggccggggcccgcgagggcgccgagcgcggccucagcgccauccgcgagcgccuggggccccugguggaacagggccgcgugcgggccgccacugugggcucccuggccggccagccgcuacaggagcgggcccaggccuggggcgagcggcugcgcgcgcggauggaggagaugggcagccggacccgcgaccgccuggacgaggugaaggagcagguggcggaggugcgcgccaagcuggaggagcaggcccagcagauacgccugcaggccgaggccuuccaggcccgccucaagagcugguucgagccccugguggaagacaugcagcgccagugggccgggcugguggagaaggugcaggcugccgugggcaccagcgccgccccugugcccagcgacaaucacugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgcaaaaaaaaaaaaaaaaaaaaaaaaaaaaaaaaaaaaaaaaaaaac

SEQ ID NO: 12 - DNA, 1166 bp, homo sapien
ctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 13 - DNA, 1181 bp, homo sapien
ctactcagccccagcggaggtgaaggacgtccttccccaggagccgtttctccttccccagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 14 - DNA, 1248 bp, homo sapien
ctactcagccccagcggaggtgaaggacgtccttccccaggagccggtgagaagcgcagtcgggggcacggggatgagctcaggggcctctagaaagagctgggaccctgggaacccctggcctccagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 15 - DNA, 1144 bp, homo sapien
agagacgacccgacccgctagaagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 16 - DNA, 1265 bp, homo sapien
ggatggggagataagagaagaccaggagggagttaaatagggaatgggttgggggcggcttggtaaatgtgctgggattaggctgttgcagataatgcaacaaggcttggaaggctaacctgggactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 17 - DNA, 1208 bp, homo sapien
agggggagccctataattggacaagtctgggatccttgagtcctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 18 - DNA, 1157 bp, homo sapien
ccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaagtcacccaagaactgagggcgctgatggacgagaccatgaaggagttgaaggcctacaaatcggaactggaggaacaactgaccccggtagcggaggagacgcgggcacggctgtccaaggagctgcagacggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatcccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagtcggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 19 - DNA, 1186 bp, homo sapien
ggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaaaaaaaaaaaaaaaaaaaaaaaaaaaac

SEQ ID NO: 20 - protein, 317 aa, homo sapien [sequence omitted]

SEQ ID NO: 21 - protein, 343 aa, homo sapien
MSSGASRKSWDPGNPWPPDWPITGRKMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 22 - DNA, 20 bp, artificial sequence
  gRNA Recognition Sequence
gactacaggcgcataccact

SEQ ID NO: 23 - DNA, 20 bp, artificial sequence
  gRNA Recognition Sequence
gcataccactaggattaatt

SEQ ID NO: 24 - DNA, 20 bp, artificial sequence
  gRNA Recognition Sequence
cccccccaaattaatcctag

SEQ ID NO: 25 - DNA, 20 bp, artificial sequence
  gRNA Recognition Sequence
gcgcctgtagtctcagctac

SEQ ID NO: 26 - DNA, 20 bp, artificial sequence
  gRNA Recognition Sequence
accactaggattaatttggg

SEQ ID NO: 27 - DNA, 20 bp, artificial sequence
  gRNA Recognition Sequence
atcacttatcacttgagtcc

SEQ ID NO: 28 - DNA, 20 bp, artificial sequence
  gRNA Recognition Sequence
tttccagtagctgagactac

SEQ ID NO: 29 - DNA, 20 bp, artificial sequence
  gRNA Recognition Sequence
ccactaggattaatttgggg

SEQ ID NO: 30 - DNA, 20 bp, artificial sequence
  gRNA Recognition Sequence
acttatcacttgagtccagg

SEQ ID NO: 31 - DNA, 20 bp, artificial sequence
  gRNA Recognition Sequence
tgggagtatcgcttgagccc

SEQ ID NO: 32 - DNA, 20 bp, artificial sequence
  gRNA Recognition Sequence
taccactaggattaatttgg

SEQ ID NO: 33 - DNA, 20 bp, artificial sequence
  gRNA Recognition Sequence
ccaggaattccacatcagcc

SEQ ID NO: 34 - DNA, 20 bp, artificial sequence
  gRNA Recognition Sequence
cataccactaggattaattt

SEQ ID NO: 35 - DNA, 20 bp, artificial sequence
  gRNA Recognition Sequence
tgtagtctcagctactggaa

SEQ ID NO: 36 - DNA, 20 bp, artificial sequence
  gRNA Recognition Sequence
ccaggctgatgtggaattcc

SEQ ID NO: 37 - DNA, 20 bp, artificial sequence
  gRNA Recognition Sequence
cactaggattaatttggggg

SEQ ID NO: 38 - DNA, 20 bp, artificial sequence
  gRNA Recognition Sequence
ataccactaggattaatttg

SEQ ID NO: 39 - DNA, 20 bp, artificial sequence
  gRNA Recognition Sequence
ggggtctggctttgttggcc